SEQUENCE LISTING (6 sequences)

SEQ ID NO: 1 - DNA, 3345 bp, Homo sapiens
agacgaggaacgtggagtgctcgaagtgggcgggcgtagggggctcctttgtctattgttgcaggggctttgcaacctcttgaggacccgtgaaagaccccttaaagagggtcttgagcaaagtgctgtcctgcgctttgggaagagttgcttgctttcgtttcaacccatggtttatgtttgatctttactttgctgtcatcgcgtgcaggtggctttatgcaaagggagagttctggttgacacaaatgcccagacagctagagaaatctctgagtggcgcctaagtgggccgagtctccgtgcgcttgggtggtttcactgtcagagctgaggcgctctcagacacctcacaggccattcagaatgtatgtgaaactccaactccaacgactgtgctacgtggttgaaggatataaccatcatttgcttgaaaattgtgtacattcaaacttgttcttgaaaaaaagctctctggagctcaagagtggatttgggtcttgggctaaggagcagattttcttctgaactggggcttttgcatattgaattgttccttttccatttgggactctgaagaatttgagaggggtagaatttctgaatcagacattacaaagactttgtgtatgtatttttaccatcagttttgtccagaaaagtgaacctggtcaatggattatttatgagcctgctagctaactctcatcataggaagacgagagcaaatatttaatgtgttttgccttcagttacataggaaaaatgtctaagaaggtgacattgggacttgtgtttaatgaaacagagaactgtgggcagcgtcagtgttgggtttttaggagcgtagggagcacacagctttgactctttgtcccattacttgcttctgtgtgaagccactgaggccccagggttcgggttttcctgccacgcactctggggtggcagtgaccgttccaatatggatgagtgagaagcaggttcttatggggtttgtgcaaattgagcaagccacttggggcatgggtttttcctcttttctttctttctttctttttcccactaaagaacagattgaaagtgcttcacaattaatcaaaagtcccctcaacactctggtgatccatctaagacctctcagagatataaccaccagcacagatattcaaacccatttttttcaatctccttgtttaagaaatatatatatactaggagtgggatgggggaataatgttgatgttttaatggaatctgaaagacctcagaaattcctctcgggggatgtttagagtcgtctgagcattttgatgaaacttggagaggcatgttgaagagtttcgcaaataggtgaggtgcctgaggaatggggatgcttggctggaggggggttaggagcatggatcagtctgtttcttacagcaggtgcttgcttcccaaacaccctggatgtccgctgccaaacagaaacacaggtaactccagacattaagccaatacgtggcagctccttctccttcctagttgtggaatgaatggtataattggagagaatatagaaggcagggcttcctgaagaagcttaattttaatttggcttcatgaatcaacaggatatggtcatgacttgactttttctaaagatgtctggacagccagagcaaaaattatacagtcacttttggttgttgttgagaaggtaactcagaccttagttattcccagtaacatatcagctcctcgtggtcctcagctctaaatgaggggctgtggagaggactgcctaacacgatgtccgagtcccctccaactcactctgcaaccatgaccctaacctctccactttaagaaaacatgagttgaaatgatggtgcaaaaatgccggagggccggtactctctacccagatatcaccttcaaatcagggtctcctggggatcccctcaggtttcactctaaggcagaggctttgcacactctgacacatgtaagtaagttaacccctgcaccttaaccacagccaaccaacaggggcccaataacccgactccccagctgcccaaatgccctttgcaagatgcccagggtgtggtgcggtagaaatagcccccaggcagcacaaatttgggctgcaccgattttcctgtccagtccctgggctattaacagccacctccactccctgatgtttgcggtgttccatggaacatacgtgggtgtggagcgaaacatctcataaaagcgcctgtttctccacacttgggacaagtgaggcagactcccttgctcttacagggaatctgtttctgggcttaaccaggcaaaatgacattccaagggaagaaaactcccatctttctaggagggacccaggggaaaacagatgaaactagcctttcacagaaacctggcatcaatgatcttccctgagcagaaaggcaaattgctcttccaggtgggcacgggctccaccattggcctgcacagccccgtacttccaggtggagctcacggtgctgactttggttcacattcagaaatgttgccctgtgagaaacgtctctgcaggggctgctcccttcagcctgacttttgtttgctctgggtgactgggaagacgaatgctcatttactgaatgccagttcttcattccttcattgtggaagtctttattgtgagactctggggacggagaggaattagacaagggcctctaagctgagctcacatcccagccggtcagtcagataaacgcatgggtatcgagtactgctaggtcccaggaagaaagagagagcagctttcgggatggggacgatggggaggtgtccgaggtcaagagaaagcggcacgagcagacccctgtgtgccgtcctgtgggcgcggggcggcaggggaggcgcacacctgctcctttgtgcagcctcccccctcccgcaaagttaaagagcaggaaagtcaggattcctcgctcggccctgccctgccggctgctccgcgctccgctcctccctgcgagcgtgtgtgtgtgtcgggggtccctcccctcctggctctggggtcgggcgcgcaccccgccccgtagcgcggcccctccctggcgagcgcaaccccatccagcgggagcgcggagccgcggccgcggggaagcattaagtttattcgcctcaaagtgacgcaaaaattcttcaagagctctttggcggcggctatctagagatcagaccatgtgagggcccgcgggtacaaatacggccgcgccggcgcccctccgcacagccagcgccgccgggtgcctcgagggcgcgaggccagcccgcctgcccagcccgggaccagcc

SEQ ID NO: 2 - DNA, 3345 bp, Homo sapiens; identical to SEQ ID NO: 1

SEQ ID NO: 3 - DNA, 703 bp, Homo sapiens
ccagttcttcattccttcattgtggaagtctttattgtgagactctggggacggagaggaattagacaagggcctctaagctgagctcacatcccagccggtcagtcagataaacgcatgggtatcgagtactgctaggtcccaggaagaaagagagagcagctttcgggatggggacgatggggaggtgtccgaggtcaagagaaagcggcacgagcagacccctgtgtgccgtcctgtgggcgcggggcggcaggggaggcgcacacctgctcctttgtgcagcctcccccctcccgcaaagttaaagagcaggaaagtcaggattcctcgctcggccctgccctgccggctgctccgcgctccgctcctccctgcgagcgtgtgtgtgtgtcgggggtccctcccctcctggctctggggtcgggcgcgcaccccgccccgtagcgcggcccctccctggcgagcgcaaccccatccagcgggagcgcggagccgcggccgcggggaagcattaagtttattcgcctcaaagtgacgcaaaaattcttcaagagctctttggcggcggctatctagagatcagactatgtgagggcccgcgggtacaaatacggccgcgccggcgcccctccgcacagccagcgccgccgggtgcctcgagggcgcgaggccagcccgcctgcccagcccgggaccagcc

SEQ ID NO: 4 - DNA, 2521 bp, Homo sapiens
ttgcaacctcttgaggacccgtgaaagaccccttaaagagggtcttgagcaaagtgctgtcctgcgctttgggaagagttgcttgctttcgtttcaacccatggtttatgtttgatctttactttgctgtcatcgcgtgcaggtggctttatgcaaagggagagttctggttgacacaaatgcccagacagctagagaaatctctgagtggcgcctaagtgggccgagtctccgtgcgcttgggtggtttcactgtcagagctgaggcgctctcagacacctcacaggccattcagaatgtatgtgaaactccaactccaacgactgtgctacgtggttgaaggatataaccatcatttgcttgaaaattgtgtacattcaaacttgttcttgaaaaaaagctctctggagctcaagagtggatttgggtcttgggctaaggagcagattttcttctgaactggggcttttgcatattgaattgttccttttccatttgggactctgaagaatttgagaggggtagaatttctgaatcagacattacaaagactttgtgtatgtatttttaccatcagttttgtccagaaaagtgaacctggtcaatggattatttatgagcctgctagctaactctcatcataggaagacgagagcaaatatttaatgtgttttgccttcagttacataggaaaaatgtctaagaaggtgacattgggacttgtgtttaatgaaacagagaactgtgggcagcgtcagtgttgggtttttaggagcgtagggagcacacagctttgactctttgtcccattacttgcttctgtgtgaagccactgaggccccagggttcgggttttcctgccacgcactctggggtggcagtgaccgttccaatatggatgagtgagaagcaggttcttatgaggtttgtgcaaattgagcaagccacttggggcatgggtttttcctcttttctttctttctttctttttcccactaaagaacagattgaaagtgcttcacaattaatcaaaagtcccctcaacactctggtgatccatctaagacctctcagagatataaccaccagcacagatattcaaacccatttttttcaatctccttgtttaagaaatatatatatactaggagtgggatgggggaataatgttgatgttttaatggaatctgaaagacctcagaaattcctctcgggggatgtttagagtcgtctgagcattttgatgaaacttggagaggcatgttgaagagtttcgcaaataggtgaggtgcctgaggaatggggatgcttggctggaggggggttaggagcatggatcagtctgtttcttacagcaggtgcttgcttcccaaacaccctggatgtccgctgccaaacagaaacacaggtaactccagacattaagccaatacgtggcagctccttctccttcctagttgtggaatgaatggtataattggagagaatatagaaggcagggcttcctgaagaagcttaattttaatttggcttcatgaatcaacaggatatggtcatgacttgactttttctaaagatgtctggacagccagagcaaaaattatacagtcacttttggttgttgttgagaaggtaactcagaccttagttattcccagtaacatatcagctcctcgtggtcctcagctctaaatgaggggctgtggagaggactgcctaacacgatgtccgagtcccctccaactcactctgcaaccatgaccctaacctctccactttaagaaaacatgagttgaaatgatggtgcaaaaatgccggagggccggtactctctacccagatatcaccttcaaatcagggtctcctggggatcccctcaggtttcactctaaggcagaggctttgcacactctgacacatgtaagtaagttaacccctgcaccttaaccacagccaaccaacaggggcccaataacccgactccccagctgcccaaatgccctttgcaagatgcccagggtgtggtgcggtagaaatagcccccaggcagcacaaatttgggctgcaccgattttcctgtccagtccctgggctattaacagccacctccactccctgatgtttgcggtgttccatggaacatacgtgggtgtggagcgaaacatctcataaaagcgcctgtttctccacacttgggacaagtgaggcagactcccttgctcttacagggaatctgtttctgggcttaaccaggcaaaatgacattccaagggaagaaaactcccatctttctaggagggacccaggggaaaacagatgaaactagcctttcacagaaacctggcatcaatgatcttccctgagcagaaaggcaaattgctcttccaggtgggcacgggctccaccattggcctgcacagccccgtacttccaggtggagctcacggtgctgactttggttcacattcagaaatgttgccctgtgagaaacgtctctgcaggggctgctcccttcagcct

SEQ ID NO: 5 - DNA, 1119 bp, Homo sapiens
acatgtgggcctacgcacatctttgctgagggagtgcttctccatgaagaaggccagattccatgtttactgcttcgggcggcaccagcgttccataattcccatcactcttgggagatgccctgggaacttcagcctccctctgaatagaggatacgaactgccagttggctctatgacctgtaactaatcctatctcaacgttaaacccacagttcatttgtttttgtttgttttgagaaagaaagttgagtttcctgagaaatgaactcactttgatcttgacacatgaagtcaaagttcaacatttctgtagtgtctttctgcttagaaggcactgataggcatcaagtagatggagcgtcacaggcatttccaaagggctgggtgtttatgaccaatcgggataattacatgcttgttcaggtacgggttgctcctggctatagagacaaagtgtgtggaaagaggaatggagattcgatcagaagtttaaatagaaacagtccttttttacttggagtcagggattgggggaatggagtgcattagcttcaggagagttaagagaaagtcaaccactcccattggctggtcaactgcacccctcatgtgcagagacctagggggacgaagcttctgtctgccagctgtcattccacagcagaggtggggtccacggcaccaccctcagcctgtcaccagctcagcctggaacaacagcacacatccaagatgcaaaggaaggcttccttctggggctttttctggacatggagaaactcaaagactgcctggagaaggtcacagctgtaatattgacctcgtttgcaatctttccacccagcaggataaaaataccttttaaaaaaagaaagcttagaagagggcattctcagaaaccaaccttcttaacaactcacacttctctattcctgggccagctcgttttcagccattgttatgtgcaagtcttgatagaaaaagccagccagggctctgcagaatcagaaaaaataggaagagagtctggggctccacaggcaggagcttgcaggcagaaagggccccgggcacatggcatcttctcctggcagacccgattagtcaccgagagga

SEQ ID NO: 6 - DNA, 1004 bp, Homo sapiens
ggcctggtaaatggttacctatggtgggtgagaggatgtgtggttattttaaaagcctatcaagcctagttgagggccgaaggaagggggaagtggggggaggccaaggccaggcagtagcataaatggtgagcagggtgaggttatggagaccgctcttgtcagaattcatatccttctgagtggtgtttgcattcttgagcctggggtgggtggcggagggcccaacaagtgcggtacagcccaagacgtctacctccccttacacacacacagcccctagctacatgaagctgatttatctcttgttgctgtagatatttttggcaacaatcgatccctttaaaatttttattttccactttacagaaaaaatgcagcaaacgccaaaccctagtgactgacattgcctggtgttcatgtctaatccttgctcatgtttatagccatagtcattcttgtgaagctactattttgtgtcctgatttttcacttactatgtttttgtcttcacatgtgtaggtagctaagttgcagtcaccaaaaaccccaaataggtaggagacatccatgcgccatttctattagccagttcctagccatcctcctaactgttggatctggagggttagcctcagatcattgcagttataaacagtccatccctggctatcttttctagaagggtccagtgtcatctcagctaggcagaaacgtatttcaactcccaaactgtttcaagagtttgaaaagctcaggcccagaagaaacagagtgagcagttgaacagtcttacaactgctgcttacaactttataacaggccataggtttaaagtccaaaagggctcaaatttctaccccacgtttgacgactcagacctcaagccagagctgagctgacttccctgcgcccaggccacggcggtgaaatgaaggcacagcagccccggccaggccctcccagtggaggcacagggggacggcgacaccaaccaccccagc

(19) United States
(12) Patent Application Publication
(10) Pub. No.: US 2015/0315609 A1
(43) Pub. Date: Nov. 5, 2015

(54) SLC6A4 MINI-PROMOTERS

(71) Applicant: The University of British Columbia, Vancouver (CA)

(72) Inventors: Elizabeth M. Simpson, Vancouver (CA); Cletus D'Souza, Vancouver (CA); Charles de Leeuw, Seattle, WA (US); Daniel Goldowitz, Port Moody (CA); Robert A. Holt, North Vancouver (CA); Steven J. Jones, Vancouver (CA); Elodie Portales-Casamar, Vancouver (CA); Wyeth W. Wasserman, Vancouver (CA); Vikramjit Chopra, Vancouver (CA)

(21) Appl. No.: 14/703,791

(22) Filed: May 4, 2015

Related U.S. Application Data
(60) Provisional application No. 61/988,778, filed on May 5, 2014.

(51) Int. Cl. C12N 15/85

(52) U.S. Cl. CPC C12N 15/85

(57) ABSTRACT

Isolated polynucleotides comprising a SLC6A4 mini-promoters are provided. The mini-promoter may be operably linked to an expressible sequence, e.g. reporter genes, genes encoding a polypeptide of interest, regulatory RNA sequences such as miRNA, siRNA, anti-sense RNA, etc., and the like. In some embodiments a cell comprising a stable integrant of an expression vector is provided, which may be integrated in the genome of the cell. The promoter may also be provided in a vector, for example in combination with an expressible sequence. The polynucleotides find use in a method of expressing a sequence of interest, e.g. for identifying or labeling cells, monitoring or tracking the expression of cells, gene therapy, etc.

### FIELD OF THE INVENTION

The invention relates to gene promoters and regulatory elements. More specifically, the invention relates to novel SLC6A4 promoter compositions and related methods.

### BACKGROUND

The solute carrier family 6 (neurotransmitter transporter), member 4 gene (SLC6A4) encodes the serotonin or 5-hydroxytryptamine transporter (also known as SERT). In the brain, SLC6A4 is expressed in regions containing serotonergic neurons, including the raphe nuclei (Lebrand et al. 1998). In situ hybridization in mouse further demonstrated expression in the raphe nuclei of the mouse midbrain (Chang et al. 1996). Similarly, a SERT-cre knock-in mouse was shown to express in the raphe nuclei (Zhuang et al. 2005).

There is a need for characterized human SLC6A4 promoters for gene expression, for instance in human gene therapy applications. It is particularly useful to identify small promoter elements that are sufficient to drive expression in regions of the brain, for instance in the raphe nuclei on serotogenic nerve terminals, as well as in the thalamus, cortex, olfactory bulb, and any scattered serotonergic system cells in the central nervous system. Such small promoter elements, or “mini-promoters” are particularly useful in certain applications, for instance they are more amenable to insertion into viral vectors used in gene therapy applications.

SLC6A4 promoter elements described in the art, including:

Lebrand C, Cases O, Wehrlé R, Blakely R D, Edwards R H, Gaspar P. Transient developmental expression of monoamine transporters in the rodent forebrain. J Comp Neurol. 1998 Nov. 30; 401(4):506-24.

Bradley C C, Blakely R D. Alternative splicing of the human serotonin transporter gene. J Neurochem. 1997 October; 69(4):1356-67.

Ozsarac N, Santha E, Hoffman B J. Alternative non-coding exons support serotonin transporter mRNA expression in the brain and gut. J Neurochem. 2002 July; 82(2):336-44.

Chang A S, Chang S M, Starnes D M, Schroeter S, Bauman A L, Blakely R D. Cloning and expression of the mouse serotonin transporter. Brain Res Mol Brain Res. 1996 Dec. 31; 43(1-2):185-92.

Zhuang X, Masson J, Gingrich J A, Rayport S, Hen R. Targeted gene expression in dopamine and serotonin neurons of the mouse brain. J Neurosci Methods. 2005 Apr. 15; 143(1):27-32.

Heils A, Wichems C, Mossner R, Petri S, Glatz K, Bengel D, Murphy D L, Lesch K P. Functional characterization of the murine serotonin transporter gene promoter in serotonergic raphe neurons. J Neurochem. 1998 March; 70(3):932-9.

### SUMMARY OF THE INVENTION

The present invention provides novel nucleic acid sequence compositions and methods relating to minimal human SLC6A4 promoters. The invention is based in part on the surprising discovery that certain minimal SLC6A4 promoter elements are capable of expressing in specific cell types, for instance in cells of the brain.

In one embodiment of the invention, there is provided an isolated nucleic acid fragment comprising a SLC6A4 mini-promoter, wherein the SLC6A4 mini-promoter comprises a SLC6A4 regulatory element operably linked in a non-native conformation to a SLC6A4 basal promoter. The SLC6A4 mini-promoter may have a nucleic acid sequence which is substantially similar in sequence and function to SEQ ID NO: 1 or 2. The SLC6A4 basal promoter may have a nucleic acid sequence which is substantially similar in sequence and function to SEQ ID NO: 3. The SLC6A4 regulatory element may have a nucleic acid sequence which is substantially similar in sequence and function to SEQ ID NO: 4, 5 or 6. In other embodiments, there is provided an isolated nucleic acid fragment comprising a SLC6A4 mini-promoter, wherein the SLC6A4 mini-promoter comprises a SLC6A4 basal promoter. The SLC6A4 basal promoter may have a nucleic acid sequence which is substantially similar in sequence and function to SEQ ID NO: 3 The SLC6A4 mini-promoters may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule.

In one embodiment, there is provided an expression vector comprising a SLC6A4 mini-promoter, wherein the SLC6A4 mini-promoter comprises a SLC6A4 regulatory element operably linked in a non-native conformation to a SLC6A4 basal promoter. The SLC6A4 mini-promoter may have a nucleic acid sequence which is substantially similar in sequence and function to SEQ ID NO: 1 or 2. The SLC6A4 basal promoter may have a nucleic acid sequence which is substantially similar in sequence and function to SEQ ID NO: 3. The SLC6A4 regulatory element may have a nucleic acid sequence which is substantially similar in sequence and function to SEQ ID NO: 4, 5 or 6. In other embodiments, there is provided an expression vector comprising a SLC6A4 mini-promoter, wherein the SLC6A4 mini-promoter comprises a SLC6A4 basal promoter. The SLC6A4 basal promoter may have a nucleic acid sequence which is substantially similar in sequence and function to SEQ ID NO: 3. The SLC6A4 mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule. The expression vector may further comprise a genomic targeting sequence. The genomic targeting sequence may be HPRT.

In one embodiment, there is provided a method for expressing a gene, protein, RNA interference molecule or the like in a cell, the method comprising introducing into the cell an expression vector comprising a SLC6A4 mini-promoter element, wherein the SLC6A4 mini-promoter element comprises a SLC6A4 regulatory element operably linked in a non-native conformation to a SLC6A4 basal promoter element. In another embodiment, the SLC6A4 mini-promoter comprises a SLC6A4 basal promoter. Cells of interest include, without limitation, cells of the peripheral or central nervous system and progenitors thereof, e.g. embryonic stem cells, neural stem cells, neurons, glial cells, astrocytes, microgial cells, etc. The SLC6A4 mini-promoter may have a nucleic acid sequence which is substantially similar in sequence and function to SEQ ID NO: 1-3. The SLC6A4 regulatory element may have a nucleic acid sequence which is substantially similar in sequence and function to SEQ ID NO: 4, 5 or 6. The SLC6A4 basal promoter may have a nucleic acid sequence which is substantially similar in sequence and function to SEQ ID NO: 3. The SLC6A4 mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule. The expression vector may thus further comprise a genomic targeting sequence. The genomic targeting sequence may be HPRT.

In one embodiment of the invention, there is provided a method for identifying or labeling a cell, the method comprising introducing into the cell an expression vector comprising a SLC6A4 mini-promoter element, wherein the SLC6A4 mini-promoter element comprises a SLC6A4 regulatory element operably linked in a non-native conformation to a SLC6A4 basal promoter element, and wherein the expressible sequence comprises a reporter gene. In other embodiments, the SLC6A4 mini-promoter comprises a SLC6A4 basal promoter. The SLC6A4 mini-promoter element may have a nucleic acid sequence substantially similar in sequence and function to SEQ ID NO: 1-3. The SLC6A4 regulatory element may have a nucleic acid sequence substantially similar in sequence and function to SEQ ID NO: 4, 5 or 6. The SLC6A4 basal promoter element may have a nucleic acid sequence substantially similar in sequence and function to SEQ ID NO: 3. In some embodiments, the cell is a peripheral or central nervous system cell or progenitors thereof, including, without limitation, embryonic stem cells, neural stem cells, glial cells, astrocytes, neurons and the like etc. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, RNA interference molecule and the like.

In one embodiment of the invention, there is provided a method for monitoring or tracking the development or maturation of a cell, the method comprising: 1) introducing into the cell a expression vector comprising a SLC6A4 mini-promoter element operably linked to an expressible sequence, wherein the SLC6A4 mini-promoter element comprises a SLC6A4 regulatory element operably linked in a non-native conformation to a SLC6A4 basal promoter element, and wherein the expressible sequence comprises a reporter gene; and 2) detecting the expression of the reporter gene in the cell of in progeny of the cell as a means of determining the lineage, identity or developmental state of the cell or cell progeny. In other embodiments, the SLC6A4 mini-promoter comprises a SLC6A4 basal promoter. The SLC6A4 mini-promoter element may have a nucleic acid sequence substantially similar in sequence and function to SEQ ID NO: 1-3. The SLC6A4 regulatory element may have a nucleic acid sequence substantially similar in sequence and function to SEQ ID NO: 4, 5 or 6. The SLC6A4 basal promoter element may have a nucleic acid sequence substantially similar in sequence and function to SEQ ID NO: 3. In some embodiments, the cell is a peripheral or central nervous system cell or progenitors thereof, including, without limitation, embryonic stem cells, neural stem cells, glial cells, neurons and the like.

In certain embodiments of the invention, there is thus provided a method of treatment of a subject having a disease involving the serotonergic system, the method comprising administering to the subject a therapeutically effective dose of a composition comprising a SLC6A4 mini-promoter element, wherein the SLC6A4 mini-promoter element comprises a SLC6A4 regulatory element operably linked in a non-native conformation to a SLC6A4 basal promoter element. In another embodiment, the SLC6A4 mini-promoter comprises a SLC6A4 basal promoter. The SLC6A4 mini-promoter element may have a nucleic acid sequence substantially similar in sequence and function to SEQ ID NO: 1-3. The SLC6A4 regulatory element may have a nucleic acid sequence substantially similar in sequence and function to SEQ ID NO: 4, 5 or 6. The SLC6A4 basal promoter element may have a nucleic acid sequence substantially similar in sequence and function to SEQ ID NO: 3. The disease or condition may be chosen from: depression, anxiety, obsessive-compulsive disorder, addiction, Huntington's disease, Parkinsons, or any neurological disorder that may benefit from expression in serotonergic cells.

### SHORT DESCRIPTION OF FIGURES

The patent or application file contains at least one drawing executed in color. Copies of this patent or patent application publication with color drawing(s) will be provided by the Office upon request and payment of the necessary fee.

FIG. 1—DNA expression vector (pEMS1136) into which SLC6A4 promoter elements were inserted for expression studies. The SLC6A4 promoter with a nucleic acid sequence corresponding to SEQ ID NO: 1 was inserted into the multiple cloning site (MCS) of the pEMS1136 vector such that it became operably linked to the lac Z reporter gene also contained the HPRT genomic targeting sequence, an ampicillin resistance gene (AmpR) for screening, and a transcriptional termination sequence (SV40 polyA), as well as other elements necessary for vector replication and gene expression.

FIG. 2A-2E—The Ple198 construct (SLC6A4-based MiniPromoter) is expressed in the thalamus. Mice were injected with AAV9 viruses intravenously carrying either the Ple198-icre (FIG. 2A, FIG. 2C) or the Promoterless-icre construct FIG. 2B. Mice were harvested via perfusion and stained overnight for lacZ activity (blue), indicative of recombination of the Gt(ROSA26)Sor^{tm1Sor} locus driven by the iCre recombinase at postnatal day 56 (P56). FIG. 2C—The promoterless construct showed minimal expression in the thalamus. FIG. 2D—An in situ hybridization image from the Allen Brain Atlas at P4 demonstrates that the Ple198 expression matches that of the source gene, SLC6A4. FIG. 2E—In contrast to P4, by P14 the endogenous gene no longer expresses in the thalamus. FIG. 2A, coronal; (FIG. 2B-2E), sagittal. LGd, dorsal lateral geniculate; LGv, ventral lateral geniculate; Mb, midbrain; Th, thalamus.

FIG. 3A-3D—Mice carrying Hprt knock-ins of the Ple198 (SLC6A4-based) MiniPromoter expresses in the retina and brain. The Ple198-icre/ERT2 construct was knocked-in at the Hprt locus in mouse embryonic stem cells and germline mice were established. FIG. 3A—The allele drives expression of the icre recombinase along with a tamoxifen-inducible ER^{T2} coding sequence. The ER^{T2} sequence can be deleted using a FLP recombinase, which excises the segments between the two F3 FRT sites. FIG. 3B—These mice were then crossed with the Ai14 strain, which contains a lox-STOP-lox tdTomato under the expression of a ubiquitous promoter. Upon recombination of the lox sites by the icre recombinase, strong expression of the red fluorescent protein, tdTomato, is observed. FIG. 3C—Adult mice were harvested via perfusion, cryosectioned, and imaged using confocal microscopy. DAPI staining in blue indicates nuclei, while tdTomato signal in red indicates indirect expression of Ple198 during tamoxifen induction. FIG. 3D—Representative images of eyes and brains of mice expressing Ple198-icre (not induced by tamoxifen). CAG Pr, CAG promoter; Ctx, cortex; ER^{T2}, modified estrogen receptor; FRT, flp recombinase target; GCL, ganglion cell layer; Hipp, hippocampus; I-SceI, restriction enzyme cut site; INL, inner nuclear layer; lox, icre recombination site; Mb, midbrain; Ob, olfactory bulb; ONL, outer nuclear layer; RRs, regulatory regions; Str, striatum; SV40 pA, SV40 poly-adenylation signal; Th, thalamus; WPRE, woodchuck post-transcriptional regulatory element.

### DETAILED DESCRIPTION

The compositions of the present invention include novel polynucleotides comprising SLC6A4 promoter elements (also referred to herein as SLC6A4 mini-promoters) as well as novel expression vectors comprising said SLC6A4 promoter elements (or mini-promoters). The present invention also includes various methods utilizing these novel SLC6A4 promoter (or mini-promoter) elements or expression vectors.

The term ‘SLC6A4’ refers to the gene which encodes the SLC6A4 protein, other aliases included the Serotonin Reuptake Transporter (SERT) or SERT1 or the serotonin transport protein 5-hydroxy-tryptamine or 5-HTT. The human homolog of SLC6A4 is encoded by the human gene identified as EntrezGene #6532 and is located on chromosome 17 at location 17q11.2. The protein encoded by human SLC6A4 has the Protein Accession NP_{—}001036 however other protein accession numbers may also be assigned to this protein. SLC6A4 may also include other isoforms and/or splice variants. Other mammalian SLC6A4 homologs may include but are not limited to: Rattus norvegicus (EntrezGene #25553), Mus musculus (EntrezGene #15567).

The term ‘promoter’ refers to the regulatory DNA region which controls transcription or expression of a gene and which can be located adjacent to or overlapping a nucleotide or region of nucleotides at which RNA transcription is initiated. A promoter contains specific DNA sequences which bind protein factors, often referred to as transcription factors, which facilitate binding of RNA polymerase to the DNA leading to gene transcription. A ‘basal promoter’, also referred to as a ‘core promoter’, usually means a promoter which contains all the basic necessary elements to promote transcriptional expression of an operably linked polynucleotide. Eukaryotic basal promoters typically, though not necessarily, contain a TATA-box and/or a CAAT box. A ‘SLC6A4 basal promoter’, in the context of the present invention and as used herein, is a nucleic acid compound having a sequence with at least 65%, at least 70%, at least. 80%, at least 85%, at least 90%, at least 95%, or at least 99% similarity to SEQ ID NO: 3.

A promoter may also include ‘regulatory elements’ that influence the expression or transcription by the promoter. Such regulatory elements encode specific DNA sequences which bind other factors, which may include but are not limited to enhancers, silencers, insulators, and/or boundary elements. A ‘SLC6A4 regulatory element’, in the context of the present invention and as used herein, is a nucleic acid compound having a sequence with at least 65%, at least 70%, at least 80%, at least 85%, at least 90%, at least 95%, or at least 99% similarity to SEQ ID NO: 4, 5 or 6. The present invention provides, in certain embodiments as described herein, different promoters of the SLC6A4 gene. In some embodiments, the SLC6A4 promoter comprises a SLC6A4 regulatory element operably linked to a SLC6A4 basal promoter.

The term ‘operably linked’, in the context of the present invention, means joined in such a fashion as to work together to allow transcription. In some embodiments of the invention, two polynucleotide sequences may be operably linked by being directly linked via a nucleotide bond. In this fashion, the two operably linked elements contain no intervening sequences and in being joined are able to direct transcription of an expression sequence. In other embodiments of the invention, two elements may be operably linked by an intervening compound, for instance a polynucleotide sequence of variable length. In such a fashion, the operably linked elements, although not directly juxtaposed, are still able to direct transcription of an expression sequence. Thus, according to some embodiments of the invention, one or more promoter elements may be operably linked to each other, and additionally be operably linked to a downstream expression sequence, such that the linked promoter elements are able to direct expression of the downstream expression sequence.

The term ‘mini-promoter’ refers to a promoter in which certain promoter elements are selected from an endogenous full length promoter for a gene, usually in such a fashion as to reduce the overall size of the promoter compared to the native sequence. For example, after identification of critical promoter elements, using one or more of various techniques, the native sequences that intervene between identified elements may be partially or completely removed. Other non-native sequences may optionally be inserted between the identified promoter elements. Promoter sequences such as enhancer elements may have an orientation that is different from the native orientation—for example, a promoter element may be inverted, or reversed, from its native orientation. Alternatively, selecting a minimal basal promoter that is sufficient to drive expression in particular cells or tissues may also be desirable. Since promoter elements that impact expression patterns are known to be distributed over varying distances of the proximal and/or distal endogenous promoter, it is a non-trivial task to identify a mini-promoter comprising a minimal basal promoter and optional regulatory regions that will adequately express in the desired cell or tissue types. A mini-promoter may provide certain advantages over native promoter conformations. For example, the smaller size of the mini-promoter may allow easier genetic manipulation, for example in the design and/or construction of expression vectors or other recombinant DNA constructs. In addition, the smaller size may allow easier insertion of DNA constructs into host cells and/or genomes, for example via transfection, transformation, etc. Other advantages of mini-promoters are apparent to one of skill in the art. In some embodiments of the invention, there are thus provided novel SLC6A4 mini-promoters comprising a SLC6A4 regulatory element operably linked in a non-native conformation to a SLC6A4 basal promoter. In general the spacing between the SLC6A4 regulatory element and the SLC6A4 basal promoter is not more than about 15 KB, generally not more than about 10 KB, usually not more than about 1 KB, more often not more than about 500 nt, and may be not more than about 100 nt, down to a direct joining of the two sequences. In other embodiments, there is provided a minimal SLC6A4 basal promoter.

The term ‘expressible sequence’ refers to a polynucleotide composition which is operably linked to a promoter element such that the promoter element is able to cause transcriptional expression of the expression sequence. An expressible sequence is typically linked downstream, on the 3′-end of the promoter element(s) in order to achieve transcriptional expression. The result of this transcriptional expression is the production of an RNA macromolecule. The expressed RNA molecule may encode a protein and may thus be subsequently translated by the appropriate cellular machinery to produce a polypeptide protein molecule. In some embodiments of the invention, the expression sequence may encode a reporter protein. Alternately, the RNA molecule may be an antisense, RNAi or other non-coding RNA molecule, which may be capable of modulating the expression of specific genes in a cell, as is known in the art.

The term ‘RNA’ as used in the present invention includes full-length RNA molecules, which may be coding or non-coding sequences, fragments, and derivatives thereof. For example, a full-length RNA may initially encompass up to about 20 Kb or more of sequence, and frequently will be processed by splicing to generate a small mature RNA. Fragments, RNAi, miRNA and anti-sense molecules may be smaller, usually at least about 18 nt. in length, at least about 20 nt in length, at least about 25 nt. in length, and may be up to about 50 nt. in length, up to about 100 nt in length, or more. RNA may be single stranded, double stranded, synthetic, isolated, partially isolated, essentially pure or recombinant. RNA compounds may be naturally occurring, or they may be altered such that they differ from naturally occurring RNA compounds. Alterations may include addition, deletion, substitution or modification of existing nucleotides. Such nucleotides may be either naturally occurring, or non-naturally occurring nucleotides. Alterations may also involve addition or insertion of non-nucleotide material, for instance at the end or ends of an existing RNA compound, or at a site that is internal to the RNA (ie. between two or more nucleotides).

The term ‘nucleic acid’ as used herein includes any nucleic acid, and may be a deoxyribonucleotide or ribonucleotide polymer in either single or double-stranded form. A ‘polynucleotide’ or ‘nucleotide polymer’ as used herein may include synthetic or mixed polymers of nucleic acids, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e. g., phosphorothioates, phosphorodithioates, etc.), pendent moieties (e.g., polypeptides), and modified linkages (e.g., alpha anomeric polynucleotides, etc.). Also included are synthetic molecules that mimic polynucleotides in their ability to bind to a designated sequence via hydrogen bonding and other chemical interactions.

A ‘purine’ is a heterocyclic organic compound containing fused pyrimidine and imidazole rings, and acts as the parent compound for purine bases, adenine (A) and guanine (G). ‘Nucleotides’ are generally a purine (R) or pyrimidine (Y) base covalently linked to a pentose, usually ribose or deoxyribose, where the sugar carries one or more phosphate groups. Nucleic acids are generally a polymer of nucleotides joined by 3′ 5′ phosphodiester linkages. As used herein ‘purine’ is used to refer to the purine bases, A and G, and more broadly to include the nucleotide monomers, deoxyadenosine-5′-phosphate and deoxyguanosine-5′-phosphate, as components of a polynucleotide chain. A ‘pyrimidine’ is a single-ringed, organic base that forms nucleotide bases, such as cytosine (C), thymine (T) and uracil (U). As used herein ‘pyrimidine’ is used to refer to the pyrimidine bases, C, T and U, and more broadly to include the pyrimidine nucleotide monomers that along with purine nucleotides are the components of a polynucleotide chain.

It is within the capability of one of skill in the art to modify the sequence of a promoter nucleic acid sequence, e.g. the provided basal promoter and regulatory sequences, in a manner that does not substantially change the activity of the promoter element, i.e. the transcription rate of an expressible sequence operably linked to a modified promoter sequence is at least about 65% the transcription rate of the original promoter, at least about 75% the transcription rate of the original promoter sequence, at least about 80%, at least about 90%, at least about 95%, at least about 99%, or more. Such modified sequences would be considered to be ‘functionally similar’ or to have ‘functional similarity’ or ‘substantial functional similarity’ to the unmodified sequence. Such modifications may include insertions, deletions which may be truncation of the sequence or internal deletions, or substitutions. The level of sequence modification to an original sequence will determine the ‘sequence similarity’ of the original and modified sequences. Modification of the promoter elements of the present invention in a fashion that does not significantly alter transcriptional activity, as described above would result in sequences with ‘substantial sequence similarity’ to the original sequence i.e. the modified sequence has a nucleic acid composition that is at least about 65% similar to the original promoter sequence, at least about 75% similar to the original promoter sequence, at least about 80%, at least about 90%, at least about 95%, at least about 99%, or more similar to the original promoter sequence. Thus, mini-promoter elements which have substantial functional and/or sequence similarity are herein described and are within the scope of the invention.

An ‘RNA interference molecule’, or ‘RNA interference sequence’ as defined herein, may include, but is not limited to, an antisense RNA molecule, a microRNA molecule or a short hairpin RNA (shRNA) molecule. Typically, RNA interference molecules are capable of target-specific modulation of gene expression and exert their effect either by mediating degradation of the mRNA products of the target gene, or by preventing protein translation from the mRNA of the target gene. The overall effect of interference with mRNA function is modulation of expression of the product of a target gene. This modulation can be measured in ways which are routine in the art, for example by Northern blot assay or reverse transcriptase PCR of mRNA expression, Western blot or ELISA assay of protein expression, immunoprecipitation assay of protein expression, etc.

An ‘antisense RNA molecule’, as used herein, is typically a single stranded RNA compound which binds to complementary RNA compounds, such as target mRNA molecules, and blocks translation from the complementary RNA compounds by sterically interfering with the normal translational machinery. Specific targeting of antisense RNA compounds to inhibit the expression of a desired gene may design the antisense RNA compound to have a homologous, complementary sequence to the desired gene. Perfect homology is not necessary for inhibition of expression. Design of gene specific antisense RNA compounds, including nucleotide sequence selection and additionally appropriate alterations, are known to one of skill in the art.

The term ‘rnicroRNA molecule’, ‘rnicroRNA’ or ‘miRNA’, as used herein, refers to single-stranded RNA molecules, typically of about 21-23 nucleotides in length, which are capable of modulating gene expression. Mature miRNA molecules are partially complementary to one or more messenger RNA (mRNA) molecules, and their main function is to downregulate gene expression. Without being bound by theory, miRNAs are first transcribed as primary transcripts or pri-miRNA with a cap and poly-A tail and processed to short, 70-nucleotide stem-loop structures known as pre-miRNA in the cell nucleus. This processing is performed in animals by a protein complex known as the Microprocessor complex, consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end. The remaining strand, known as the anti-guide or passenger strand, is degraded as a RISC complex substrate. After integration into the active RISC complex, miRNAs base pair with their complementary mRNA molecules and induce mRNA degradation by argonaute proteins, the catalytically active members of the RISC complex. Animal miRNAs are usually complementary to a site in the 3′ UTR whereas plant miRNAs are usually complementary to coding regions of mRNAs.

The term ‘short hairpin RNA’ or ‘shRNA’ refers to RNA molecules having an RNA sequence that makes a tight hairpin turn that can be used to silence gene expression via RNA interference. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA Polymerase III whereas miRNA is transcribed by RNA Polymerase II. Techniques for designing target specific shRNA molecules are known in the art.

An ‘expression vector’ is typically a nucleic acid molecule which may be integrating or autonomous, (i.e. self-replicating), and which contains the necessary components to achieve transcription of an expressible sequence in a target cell, when introduced into the target cell. Expression vectors may include plasmids, cosmids, phage, YAC, BAC, mini-chromosomes, viruses, e.g. retroviruses, adenovirus, lentivirus, SV-40, and the like; etc. Many such vectors have been described in the art and are suitable for use with the promoters of the present invention. Expression vectors of the present invention include a promoter as described herein, operably linked to an expressible sequence, which may also be optionally operably linked to a transcription termination sequence, such as a polyadenylation sequence. The expression vector optionally contains nucleic acid elements which confer host selectivity, elements that facilitate replication of the vector, elements that facilitate integration of the vector into the genome of the target cell, elements which confer properties, for example antibiotic resistance, to the target cell which allow selection or screening of transformed cells and the like. Techniques and methods for design and construction of expression vectors are well known in the art.

It may be desirable, when driving expression of an expressible sequence with a particular promoter system to have the expression occur in a stable and consistent manner. A factor that has been shown to affect expression is the site of integration of an expression vector or construct into the genome of the target cell, sometimes called ‘position effects’. Such position effects may be caused by, for example, local chromatin structure which affects expression of sequences from that region of the genome. One method to control for position effects when integrating an expression vector or construct into the genome of a target cell is to include a ‘genomic targeting sequence’ in the vector or construct that directs integration of the vector or construct to a specific genomic site. As an example, the hypoxanthine phosphoribosyltransferase (HPRT) gene has been used successfully for this purpose (Bronson, Plaehn et al. 1996; Jasin, Moynahan et al. 1996). The HPRT gene has additional advantages as a genomic targeting sequence, for instance its concomitant use as a selectable marker system. Other genomic targeting sequences that may be useful in the present invention are described in the art, for instance (Jasin, Moynahan et al. 1996; van der Weyden, Adams et al. 2002). The genomic targeting signals as described herein are useful in certain embodiments of the present invention.

Introduction of nucleic acids or expression vectors into cells may be accomplished using techniques well known in the art, for example microinjection, electroporation, particle bombardment, or chemical transformation, such as calcium-mediated transformation, as described for example in Maniatis et al. 1982, Molecular Cloning, A laboratory Manual, Cold Spring Harbor Laboratory or in Ausubel et al. 1994, Current protocols in molecular biology, Jolm Wiley and Sons.

In certain embodiments of the invention, there are provided methods of treatment using the nucleic acids or expression vectors, for instance for gene therapy applications. The nucleic acids or expression vectors of the present invention may be administered in isolation, or may be linked to or in combination with tracer compounds, liposomes, carbohydrate carriers, polymeric carriers or other agents or excipients as will be apparent to one of skill in the art. In an alternate embodiment, such compounds may comprise a medicament, wherein such compounds may be present in a pharmacologically effective amount.

The term ‘medicament’ as used herein refers to a composition that may be administered to a patient or test subject and is capable of producing an effect in the patient or test subject. The effect may be chemical, biological or physical, and the patient or test subject may be human, or a non-human animal, such as a rodent or transgenic mouse, or a dog, cat, cow, sheep, horse, hamster, guinea pig, rabbit or pig. The medicament may be comprised of the effective chemical entity alone or in combination with a pharmaceutically acceptable excipient.

The term ‘pharmaceutically acceptable excipient’ may include any and all solvents, dispersion media, coatings, antibacterial, antimicrobial or antifungal agents, isotonic and absorption delaying agents, and the like that are physiologically compatible. An excipient may be suitable for intravenous, intraperitoneal, intramuscular, subcutaneous, intrathecal, intraocular, topical or oral administration. An excipient may include sterile aqueous solutions or dispersions for extemporaneous preparation of sterile injectable solutions or dispersion. Use of such media for preparation of medicaments is known in the art.

The nucleic acids or expression vectors of the present invention may be administered to a subject using a viral delivery system. For instance, the nucleic acids may be inserted into a viral vector using well known recombinant techniques. The subsequent viral vector may then be packaged into a virus, such as adenovirus, lentivirus, attenuated virus, adeno-associated virus (AAV), and the like. Viral delivery for gene therapy applications is well known in the art. There exist a variety of options for viruses suitable for such delivery, which may also involve selecting an appropriate viral serotype for delivery and expression in an appropriate tissue.

Compositions or compounds according to some embodiments of the invention may be administered in any of a variety of known routes. Examples of methods that may be suitable for the administration of a compound include orally, intravenous, inhalation, intramuscular, subcutaneous, topical, intraperitoneal, intra-ocular, intra-rectal or intra-vaginal suppository, sublingual, and the like. The compounds of the present invention may be administered as a sterile aqueous solution, or may be administered in a fat-soluble excipient, or in another solution, suspension, patch, tablet or paste format as is appropriate. A composition comprising the compounds of the invention may be formulated for administration by inhalation. For instance, a compound may be combined with an excipient to allow dispersion in an aerosol. Examples of inhalation formulations will be known to those skilled in the art. Other agents may be included in combination with the compounds of the present invention to aid uptake or metabolism, or delay dispersion within the host, such as in a controlled-release formulation. Examples of controlled release formulations will be known to those of skill in the art, and may include microencapsulation, embolism within a carbohydrate or polymer matrix, and the like. Other methods known in the art for making formulations are found in, for example, “Remington's Pharmaceutical Sciences”, (19th edition), ed. A. Gennaro, 1995, Mack Publishing Company, Easton, Pa.

The dosage of the compositions or compounds of some embodiments of the invention may vary depending on the route of administration (oral, intravenous, inhalation, or the like) and the form in which the composition or compound is administered (solution, controlled release or the like). Determination of appropriate dosages is within the ability of one of skill in the art. As used herein, an ‘effective amount’, a ‘therapeutically effective amount’, or a ‘pharmacologically effective amount’ of a medicament refers to an amount of a medicament present in such a concentration to result in a therapeutic level of drug delivered over the term that the drug is used. This may be dependent on mode of delivery, time period of the dosage, age, weight, general health, sex and diet of the subject receiving the medicament. Methods of determining effective amounts are known in the art. It is understood that it could be potentially beneficial to restrict delivery of the compounds of the invention to the target tissue or cell in which protein expression. It is also understood that it may be desirable to target the compounds of the invention to a desired tissue or cell type. The compounds of the invention may thus be coupled to a targeting moiety. The compounds of the invention may be coupled to a cell uptake moiety. The targeting moiety may also function as the cell uptake moiety.

### SLC6A4 Mini-Promoters

The present invention herein provides novel SLC6A4 mini-promoter sequences which are capable of effecting transcriptional expression in a spatial and temporal fashion in the brain. Certain SLC6A4 mini-promoters of the invention comprise minimal SLC6A4 promoter elements joined in a non-native configuration, thus providing advantageous characteristics. Other SLC6A4 mini-promoters of the invention comprise a minimal SLC6A4 basal promoter. Also provided are novel expression vector compositions comprising SLC6A4 mini-promoters which allow consistent specific spatiotemporal transcription of expression sequences. Also provided are novel methods utilizing these SLC6A4 mini-promoters and expression vectors.

The SLC6A4 promoters of the invention, as described herein, are referred to as ‘mini-promoters’ to reflect the fact that the mini-promoters comprise minimal SLC6A4 promoter elements sufficient to drive expression, and that may also be joined by non-native sequences. In this context, the native intervening sequences may have been partially or completely removed, and optionally may have been replaced with non-native sequences. Furthermore, the natural spatial arrangement of elements may be altered, such that downstream promoter elements (in natural conformation) are moved upstream (in non-native conformation). In such a fashion, the natural spacing of the promoter elements, for instance a human SLC6A4 regulatory element corresponding to SEQ ID NO: 4, 5 or 6 and the human SLC6A4 basal promoter elements corresponding to SEQ ID NO: 3. or sequences with substantial functional and/or sequence equivalence, is altered. Additionally, the orientation of the different promoter elements may be altered—for instance the regulatory element corresponding to SEQ ID NO: 4, 5 or 6 may be inverted relative to the basal promoter element corresponding to SEQ ID NO: 3. An advantage of such non-native mini-promoters is that the removal of native intervening sequences reduces the size of the mini-promoter while maintaining the functional activity of the promoter, thus improving the utility of the mini-promoter for various applications. Furthermore, the inversion of an enhancer/promoter element may allow retention of the enhancer properties without causing alternate promoter activity.

The inventors have demonstrated, as illustrated in the non-limiting Working Examples, that human SLC6A4 mini-promoters having a sequence corresponding to SEQ ID NO: 1 and 2 (also referred to in the Working Examples as Ple198 and Ple197), and which is comprised of one or more human SLC6A4 regulatory elements (for Ple198, the regulatory elements are SEQ ID NO: 5 and 6; for Ple197 the regulatory element is SEQ ID NO: 4) operably linked in a non-native conformation to a human SLC6A4 basal promoter having a nucleic acid sequence corresponding to SEQ ID NO: 3, is capable of directing expression of an expressible sequence which is operably linked downstream of the SLC6A4 promoter in specific cell types in different regions of the brain and/or eye. It is within the skill of one in the art to locate and determine these relative positions based on published sequence information for this gene, for instance found in the GenBank or PubMed public databases. It is understood that these genomic coordinates and relative positions are provided for the purposes of context, and that if any discrepancies exist between published sequences and the sequence listings provided herein, then the sequence listings shall prevail.

Promoters of the present invention may be modified with respect to the native regulatory and/or native basal promoter sequence. In general, such modifications will not change the functional activity of the promoter with respect to cell-type selectivity; and to the rate of transcription in cells where the promoter is active. The modified promoter provide for a transcription rate of an expressible sequence operably linked to a modified promoter sequence that is at least about 75% the transcription rate of the promoter sequence of SEQ ID NO: 1-3, at least about 80%, at least about 90%, at least about 95%, at least about 99%, or more. Methods of assessing promoter strength and selectivity are known in the art, including, for example, expression of a reporter sequence in a cell in vivo or in vitro, and quantitating the reporter activity.

Modifications of interest include deletion of terminal or internal regions, and substitution or insertion of residues. The spacing of conserved sequences may be the same as the native spacing, or it may be different than the native spacing. The order of the conserved sequences may be the same as the native order or the sequences may be rearranged. Sequences set forth in SEQ ID NO: 1-3 that are not conserved may be deleted or substituted, usually modifications that retain the spacing between conserved sequences is preferred. In general the spacing between the regulatory element and the basal promoter is not more than about 10 KB, generally not more than about 1 KB, usually not more than about 500 nt, and may be not more than about 100 nt, down to a direct joining of the two sequences.

In one embodiment of the invention, there is provided an isolated nucleic acid fragment comprising a SLC6A4 mini-promoter, wherein the SLC6A4 mini-promoter comprises a SLC6A4 regulatory element operably linked in a non-native conformation to a SLC6A4 basal promoter. The SLC6A4 mini-promoter may have a nucleic acid sequence which is substantially similar in sequence and function to SEQ ID NO: 1 or 2. The SLC6A4 basal promoter may have a nucleic acid sequence which is substantially similar in sequence and function to SEQ ID NO: 3. The SLC6A4 regulatory element may have a nucleic acid sequence which is substantially similar in sequence and function to SEQ ID NO: 4, 5 or 6. In other embodiments, there is provided an isolated nucleic acid fragment comprising a SLC6A4 mini-promoter, wherein the SLC6A4 mini-promoter comprises a SLC6A4 basal promoter. The SLC6A4 basal promoter may have a nucleic acid sequence which is substantially similar in sequence and function to SEQ ID NO: 3. The SLC6A4 mini-promoters may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule.

It is an object of the present invention to provide means of expressing a gene, protein, RNA interference molecule or the like in a cell, tissue or organ. As such, the inventors thus provide novel expression vectors comprising SLC6A4 mini-promoters which are capable of accomplishing this task. In one embodiment, there is provided an expression vector comprising a SLC6A4 mini-promoter, wherein the SLC6A4 mini-promoter comprises a SLC6A4 regulatory element operably linked in a non-native conformation to a SLC6A4 basal promoter. The SLC6A4 mini-promoter may have a nucleic acid sequence which is substantially similar in sequence and function to SEQ ID NO: 1 or 2. The SLC6A4 basal promoter may have a nucleic acid sequence which is substantially similar in sequence and function to SEQ ID NO: 3. The SLC6A4 regulatory element may have a nucleic acid sequence which is substantially similar in sequence and function to SEQ ID NO: 4, 5 or 6. In other embodiments, there is provided an expression vector comprising a SLC6A4 mini-promoter, wherein the SLC6A4 mini-promoter comprises a SLC6A4 basal promoter. The SLC6A4 basal promoter may have a nucleic acid sequence which is substantially similar in sequence and function to SEQ ID NO: 3. The SLC6A4 mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule. The expression vector may further comprise a genomic targeting sequence. The genomic targeting sequence may be HPRT, e.g. human HPRT, mouse HPRT, etc.

The inventors have herein demonstrated that expression vectors comprising novel SLC6A4 mini-promoter elements are capable of directing transcription of an expression sequence in specific cell types, for instance in neuronal cells in the brain. In one embodiment of the invention, there is thus provided a method for expressing a gene, protein, RNA interference molecule or the like in a cell, the method comprising introducing into the cell an expression vector comprising a SLC6A4 mini-promoter element, wherein the SLC6A4 mini-promoter element comprises a SLC6A4 regulatory element operably linked in a non-native conformation to a SLC6A4 basal promoter element. In another embodiment, the SLC6A4 mini-promoter comprises a SLC6A4 basal promoter. Cells of interest include, without limitation, cells of the peripheral or central nervous system and progenitors thereof, e.g. embryonic stem cells, neural stem cells, neurons, glial cells, astrocytes, microgial cells, etc.; The SLC6A4 mini-promoter may have a nucleic acid sequence which is substantially similar in sequence and function to SEQ ID NO: 1-3. The SLC6A4 regulatory element may have a nucleic acid sequence which is substantially similar in sequence and function to SEQ ID NO: 4, 5 or 6. The SLC6A4 basal promoter may have a nucleic acid sequence which is substantially similar in sequence and function to SEQ ID NO: 3. The SLC6A4 mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule. The expression vector may thus further comprise a genomic targeting sequence. The genomic targeting sequence may be HPRT.

In one embodiment of the invention, there is provided a method for identifying or labeling a cell, the method comprising introducing into the cell an expression vector comprising a SLC6A4 mini-promoter element, wherein the SLC6A4 mini-promoter element comprises a SLC6A4 regulatory element operably linked in a non-native conformation to a SLC6A4 basal promoter element, and wherein the expressible sequence comprises a reporter gene. In other embodiments, the SLC6A4 mini-promoter comprises a SLC6A4 basal promoter. The SLC6A4 mini-promoter element may have a nucleic acid sequence substantially similar in sequence and function to SEQ ID NO: 1-3. The SLC6A4 regulatory element may have a nucleic acid sequence substantially similar in sequence and function to SEQ ID NO: 4, 5 or 6. The SLC6A4 basal promoter element may have a nucleic acid sequence substantially similar in sequence and function to SEQ ID NO: 3. The inventors have demonstrated that expression vectors comprising certain human SLC6A4 promoter elements are capable of expression in specific regions of the brain. In some embodiments, the cell is a peripheral or central nervous system cell or progenitors thereof, including, without limitation, embryonic stem cells, neural stem cells, glial cell, neuronal cells, astrocytes, and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, RNA interference molecule and the like.

In further embodiments of the invention, there is provided a method for monitoring or tracking the development or maturation of a cell, the method comprising: 1) introducing into the cell a expression vector comprising a SLC6A4 mini-promoter element operably linked to an expressible sequence, wherein the SLC6A4 mini-promoter element comprises a SLC6A4 regulatory element operably linked in a non-native conformation to a SLC6A4 basal promoter element, and wherein the expressible sequence comprises a reporter gene; and 2) detecting the expression of the reporter gene in the cell of in progeny of the cell as a means of determining the lineage, identity or developmental state of the cell or cell progeny. In other embodiments, the SLC6A4 mini-promoter comprises a SLC6A4 basal promoter. The SLC6A4 mini-promoter element may have a nucleic acid sequence substantially similar in sequence and function to SEQ ID NO: 1-3. The SLC6A4 regulatory element may have a nucleic acid sequence substantially similar in sequence and function to SEQ ID NO: 4, 5 or 6. The SLC6A4 basal promoter element may have a nucleic acid sequence substantially similar in sequence and function to SEQ ID NO: 3. In such a fashion, one may be able to follow the development of a parent cell as it differentiates into more mature cells. As an example, one could introduce an expression vector comprising the aforementioned SLC6A4 mini-promoter elements into a pluripotent stem cell, monitor the expression of the reporter gene that is being expressed by the SLC6A4 promoter elements during the maturation and differentiation of the stem cell and thus determine the state of maturation, for instance in the differentiation of the pluripotent stem cell into a specific brain cell type. The inventors have demonstrated that the SLC6A4 mini-promoter elements described herein direct transcriptional expression in certain brain cell types, and so detection of reporter gene expression in a cell would thus be indicative of the cellular identity of the cell as being a certain type of brain cell.

The inventors have herein demonstrated that certain SLC6A4 mini-promoter elements of the present invention are capable of driving expression in the thalamus region of the brain. This surprising expression pattern provides additional methods of use for these mini-promoter elements. For instance, it may be desirable to utilize the SLC6A4 mini-promoters of the present invention in a gene therapy or cell therapy application wherein the SLC6A4 mini-promoters are utilized to drive expression of a therapeutic or beneficial compound, such as a protein, in neuronal cells. In such a way, the therapeutic or beneficial compound may be useful for a disease or condition that involves such neuronal cells, involves expression of a therapeutic molecule in the thalamus, or which may be improved by expression of the therapeutic or beneficial compound in those cells or other supporting cells in the central nervous system. In certain embodiments of the invention, there is thus provided a method of treatment of a subject having a disease involving the serotonergic system the method comprising administering to the subject a therapeutically effective dose of a composition comprising a SLC6A4 mini-promoter element, wherein the SLC6A4 mini-promoter element comprises a SLC6A4 regulatory element operably linked in a non-native conformation to a SLC6A4 basal promoter element. In another embodiment, the SLC6A4 mini-promoter comprises a SLC6A4 basal promoter. The SLC6A4 mini-promoter element may have a nucleic acid sequence substantially similar in sequence and function to SEQ ID NO: 1-3. The SLC6A4 regulatory element may have a nucleic acid sequence substantially similar in sequence and function to SEQ ID NO: 4, 5 or 6. The SLC6A4 basal promoter element may have a nucleic acid sequence substantially similar in sequence and function to SEQ ID NO: 3. The disease or condition may be chosen from: depression, anxiety, obsessive-compulsive disorder, addiction, Huntington's disease, Parkinsons, or any neurological disorder that may benefit from expression in serotonergic cells.

The inventors herein further describe the present invention by way of the following non-limiting examples:

### WORKING EXAMPLES

### General Methods

### Expression Vector

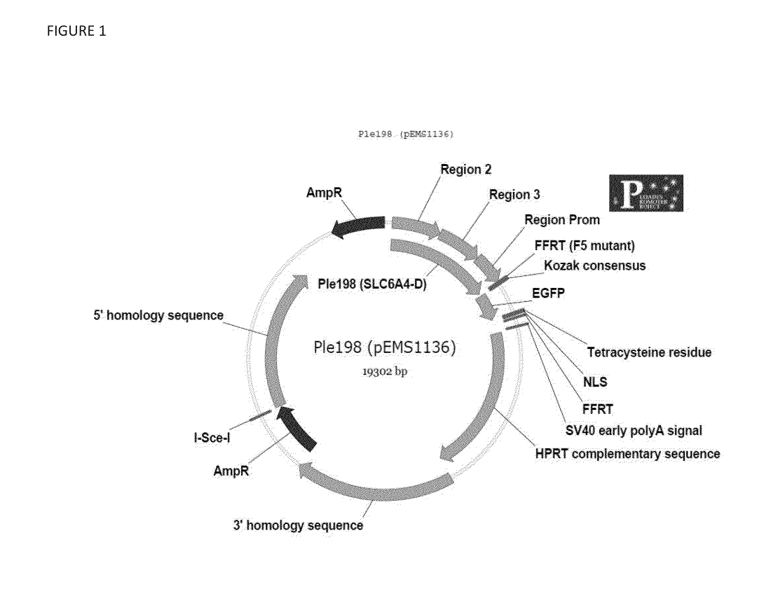

The nucleic acid fragment corresponding to SEQ ID NO: 1-6 was inserted into the multiple cloning site of pEMS1313 (driving the lacZ reporter) to produce the expression vectors that were used in the experiments. The table below (Table 1) shows the sequence identifier and construct names for each mini-promoter. FIG. 1 illustrates the design of the pEMS1136 construct containing the Ple198 mini-promoter.

| | | Corresponding | |
|---|---|---|---|
| | | nucleic acid | Size in |
| | Mini-Promoter | fragment inserted into | nucleotide |
| Construct name | Name | pEMS1313 vector | base pairs |
| pEMS1133 | SLC6A4-A (also | SEQ ID NO: 3 | 684bp |
| | referred to as | | |
| | Prom or Ple195) | | |
| pEMS1135 | SLC6A4-C (also | SEQ ID NO: 2 | 3205 |
| | referred to as | | |
| | Ple197) | | |
| pEMS1136 | SLC6A4-D (also | SEQ ID NO: 1 | 2611 |
| | referred to as | | |
| | Ple198) | | |

Blastocysts were obtained from natural mating of B6-Hprtb-m3 homozygous females to 129-ROSA26 heterozygous males at 3.5 dpc. Blastocysts were flushed from uterine horns as per (Hogan, Beddington et al. 1994), cultured in EmbryoMax® KSOM with ½ Amino Acids, Glucose and Phenol Red (Cat # MR-121, Millipore/Chermicon, Temecula, Calif.) for 3-5 h, and then transferred onto mitomycin C (mitC; Cat#M4287, Sigma, Oakville, ON) mitotically inactivated B6-Hprtb-m3, B6129F1, or 129 mouse embryonic feeders (MEFs) derived from 13.5-day post-coital embryos (Ponchio, Duma et al. 2000) in 96-well plates containing KSR-ESC (Knockout™ D-MEM, Cat#10829-018, Invitrogen, Burlington, ON) with 2 mM L-glutamine (Cat#25030-081, Invitrogen, Burlington, ON), 0.1 mM MEM nonessential amino acid solution (Cat#11140-050, Invitrogen, Burlington, ON) and 16% Knockout™ Serum Replacement (Cat#10828-028, Invitrogen, Burlington, ON)) media (MEF media was replaced 3-5 hour prior to transfer). Blastocysts were cultured as per (Cheng, Dutra et al. 2004) with the following modifications: Cells were cultured for 7-9 days in KSR-ESC with minimal disturbance (checked on day 2 to determine if the blastocysts had ‘hatched’ out of the zona pellucida) and no media changes. Blastocysts which hatched and had a well-developed ICM (inner cell mass) were treated with 20 μl 0.25% trypsin-EDTA (Invitrogen, Burlington, ON) for 5 min at 37° C., triturated with a 200 μl Pipetman, inactivated with 30 μl 0.5 mg/ml soybean trypsin inhibitor (Invitrogen, Burlington, ON), and brought up to 200 μl with KSR-ESC, then transferred individually to a 24-well MEF plate containing 1800 μl KSR-ESC, for a total volume of 2 ml. Beginning 4 days later, KSR-ESC media was replaced with FBS-ESC media (DMEM (Cat #11960-069, Invitrogen, Burlington, ON) with 2 mM L-glutamine (Invitrogen, Burlington, ON), 0.1 mM MEM nonessential amino acid solution (Invitrogen, Burlington, ON), 16% ES Cell Qualified fetal bovine serum (FBS, Invitrogen, Burlington, ON), 1000 U ESGRO-LIF (Millipore, ESG1107) and 0.01% 8-mercaptoethanol (Sigma, Oakville, ON)) in 25%, 50%, 75% proportions (respectively) to adapt the cells to FBS-containing media. On day 7 the cells were trypsinized to one well of a 24 well plate containing 1 ml of 100% FBS-ESC media, with daily media replacement. Once confluent, wells containing ESC colonies were expanded 3×24 wells (with MEFs), then passaged to 3×24 (with MEFs) and 3×12 well (plastic—no MEFs) for DNA analysis. Once confluent, the 3×24 wells were combined, aliquoted (3 vials), and frozen in ESC-freeze media (50% FBS, 40% FBS-ESC media, 10% DMSO (Sigma, Oakville, ON), and the 3×12 well treated with lysis buffer (Fisher Scientific, Ottawa, ON), mixed and aliquoted. Cultures were genotyped for X & Y chromosomes (Clapcote and Roder 2005), Gt(ROSA)26Sortm1Sor and WT alleles and Hprtb-m3 and WT alleles. B6129F1-Gt(ROSA)26Sortm1Sor/+, Hprtb-m3/Y (mEMS1204 series) and B6129F1-Gt(ROSA)26Sortm1Sor+/+, Hprtb-m3/Y (mEMS1202 series) cell lines were identified.

### Knock-in at the Hprt Locus

The expression vector plasmid DNA was purified with Qiagen Maxi Kit (Qiagen, Mississauga, ON), resuspended in 10:1 Tris-EDTA (TE, pH7.0) buffer, and linearized with I-SceI (New England Biolabs, Pickering, ON). Linearized plasmid DNA was resuspended in 85 μl of TE (10:0.1) to a final concentration of 187.5 ng/μl. Ple198 or Ple197 was targeted in our in-house derived mEMS1202 cell line. ESCs were grown to confluence on 4-6 T75 flasks of mitC treated Hprtb-m3 mouse embryonic feeders (MEFs) in FBS-ESC media. ESCs (1.7-2.5×107) in 720 μl 1×PBS were added to the linearized DNA and electroporated in a 4 mm electroporation cuvette (Bio-Rad Genepulser, Mississauga, ON), at 240 V, 50 μF, 6-10 msec pulse, immediately resuspended in a total volume of 5 ml of FBS-ESC media and plated onto 5×100 mm dishes of mitC B6129F1 MEFs in a total volume of 12 ml per 100 mm dish. 24-36 h post-electroporation, correctly targeted homologous recombinants were selected for using HAT media (FBS-ESC media containing 1×HAT ((0.1 mM sodium hypoxanthine, 0.4 mM aminopterin, 0.16 mM thymidine), Cat#21060-017, Invitrogen, Burlington, ON). HAT media was changed every day for the first 3 days, and then every 3rd day thereafter, for up to 10 days. Individual colonies were counted and, typically, no more than 2 isolated colonies were picked per 100 mm dish to optimize for independent homologous recombination events. These colonies were expanded under standard protocols for verification of the desired recombination event.

### Derivation of Knock-in Mice

Chimeric mice from targeted ESCs were generated by microinjection (Hogan, Beddington et al. 1994) into E3.5 blastocysts followed by implantation into the uterine horns of 2.5 day pseudopregnant ICR females. Chimeras were identified and coat color chimerism determined as outlined below.

Male chimeras derived from the E14TG2a cell lines were mated with B6 or B6-Alb females, and germline transmission was identified in the former case by the transmission of the dominant Aw (white bellied agouti) allele, making the progeny appear brown with a cream belly, or in the latter case by the combination of Aw and Tyrc-ch (chinchilla), making the progeny appear golden. Non-germline progeny from the cross to B6 were homozygous for the recessive a (nonagouti) allele and appeared black, whereas non-germline progeny from the cross to B6-Alb were homozygous for the recessive Tyrc-2J (albino 2 Jackson) allele and appeared white. Male chimeras derived from the cell lines were mated with B6-Alb females, and germline transmission identified by the presence of the dominant Tyr+ (tyrosinase; wild type) and the Aw (white bellied agouti) or a (nonagouti) alleles making the progeny appear brown with a cream belly or black, respectively. Non-germline progeny were homozygous for the recessive Tyrc-2J (albino 2 Jackson) allele and appeared white. All germline female offspring carry the knock-in X Chromosome and were mated with B6 males. N2 offspring were analyzed for the presence of the KI allele by PCR.

### Reporter Gene Detection

Adult male hemizygous MiniPromoter and age matched control mice were perfused with 4% paraformaldehyde (PFA) as previously described (Young, Berry et al. 2002). Whole brains were dissected out and post-perfusion immersion fixed with PFA for 2 hours at 4° C. The brains were sectioned using a coronal or sagittal brain mold (Electron Microscopy Sciences) at 1 mm and sections were placed in 12-well tissue culture plates. In brief, brain sections were rinsed with phosphate buffered saline (PBS), then incubated with X-Gal (Boeringer Mannheim, Indianapolis, Ind.) at 37° C., usually overnight. After staining the tissue was rinsed with PBS and moved into PBS containing 0.02% azide for storage. Bright field images were taken on a Leica MZ125 dissecting microscope and photographed using an Olympus Coolsnap cf color camera with the ImagePro software package.

### Example 1

### Selection of SLC6A4 Mini-Promoter Elements

Two different SLC6A4 basal promoter regions were selected and tested, while three different regulatory regions of the human SLC6A4 promoter region were selected. The basal promoters included the basal promoter of 684 bp (SEQ ID NO: 3, Ple195). Experiments also included the basal promoter (SEQ ID NO: 3) fused to regulatory region 1 (SEQ ID NO: 4) called Ple197 (SEQ ID NO: 2). Experiments also show the utility of the basal promoter (SEQ ID NO: 3) fused to regulatory regions 2 (951 bp) and 3 (976 bp) (SEQ ID NO: 5 and 6, respectively), called Ple198 (SEQ ID NO: 1). FIG. 1 shows the organization of the Ple198 construct.

### Example 2

### Expression of Reporter in Brain by the Ple198 Mini-Promoter Construct

The Ple198 construct was tested in recombinant single-stranded adeno-associated virus serotype 9 (rAAV9 or ssAAV9) driving the iCre recombinase reporter. The Ple198 expression vectors were introduced into mouse embryonic stem cells (ESCs) at the HPRT locus. The ESCs were used to generate genetically modified mice containing the SLC6A4 Mini-Promoter. Mice were injected intravenously with virus at post-natal day 0 (P0) as described elsewhere (Foust et al. 2009. Nat Biotech. 27:59-65). Expression was analyzed P21 and P56 via recombination of the reporter locus Gt(ROSA26)Sor^{tmSor1} (Soriano 1999. Nat Genetics: 21:70-71). Once recombined, this locus expressed the β-galactosidase (IacZ gene) enzyme. Histochemical reaction with the X-gal substrate results in blue signal where the reporter is expressed.

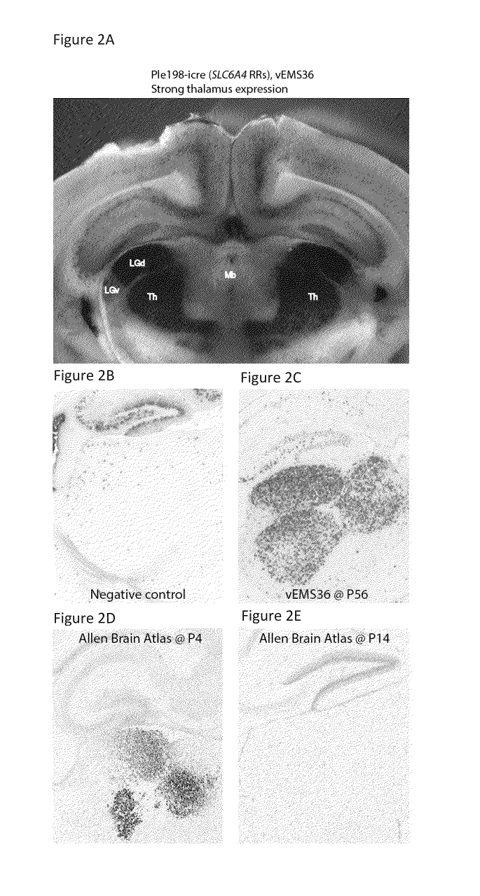

As shown in FIGS. 2A and 2C, there was strong staining identified in the thalamus as compared to the negative control in FIG. 2B. The expression observed in the coronal section (FIG. 2A) suggests there is a strong boundary between the thalamus (Th) and midbrain (Mb), and the dorsal (LGd) and ventral (LGv) regions of the lateral geniculate complex. This expression pattern matches that of the Allen Brain Atlas for SLC6A4 at P4 (FIG. 2D), but has disappeared by P14 (FIG. 2E), suggesting that this might be developmental expression captured in the adult context—an unexpected result. This expression was not observed for a promoterless virus driving iCre (FIG. 1B).

### Example 3

### Generation of an Hprt Knock-in Mouse Containing the Ple198 Construct

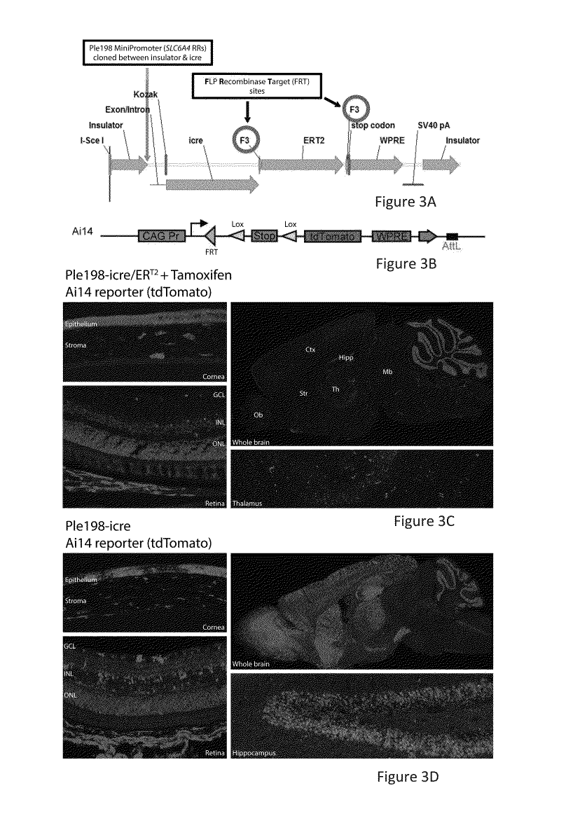

We also generated an Hprt knock-in mouse containing the Ple198 construct (FIG. 3A), driving a icre/ER^{T2} reporter (Strain name: C57BL/6-Hprt^{tm351(Ple198-icre/ERT2)Ems/Mmjax}, Jackson Laboratory Stock #037081-JAX, and also known as Ple198-icre/frt/ERT2/frt;mEMS6044, SLC6A4-creERT2). This reporter fusion consists of the icre recombinase fused to a triple mutant of the human estrogen receptor. The mutations prevent binding of endogenous estrogen, while allowing tamoxifen binding and induction. Thus, upon administration of tamoxifen, icre/ER^{T2} translocates from the cytoplasm to the nucleus and mediates recombination between IoxP sites. The Ple198-icre/ER^{T2} knock-in mice (allele tm351a) were crossed to the Ai14 reporter mouse (FIG. 3B), generated by the Allen Institute for Brain Sciences (AIBS) (Madisen et al. Nature Neurosince 2010. 13:133-140). The Ai14 mice were acquired from The Jackson Laboratory (Stock #007908, strain name: B6;129S6-Gt(ROSA)26Sor^{tm14(CAG-tdTomato)Hze}/J) Briefly, the Ai14 reporter construct was knocked into the Gt(ROSA)26Sor locus. The construct contains the tdTomato, a red fluorescent protein variant, cDNA driven by the CAG promoter. A loxP-flanked stop site between the CAG promoter and the tdTomato open reading frame prevents expression. Upon recombination of the IoxP sites (resulting in removal of the STOP fragment), the mouse expresses the red fluorescent protein molecule tdTomato from the CAG promoter in the cells that have undergone the recombination event. Therefore, in summary, upon addition of tamoxifen in either food or drinking water, the cre recombinase will be activated in cells that express the Ple198 MiniPromoter, recombine the IoxP sites, and result in tdTomato expression.

In our experiments we fed adult mice (weighing at least 20 grams) a tamoxifen-containing diet (Harlan Inc., TD.120629, 500 mg tamoxifen per kg diet, ˜80 mg tamoxifen per kg body weight per day) for a total of 4 weeks. Mice were returned to normal chow for an additional 3 weeks prior to harvesting by perfusion. Note that any cells that have undergone icre-mediated recombination will generate progeny that also contains the rearranged allele and thus express tdTomato.

In addition to the tm351a icre/ER^{T2} allele, we also generated mice that lack the ER^{T2} tamoxifen-inducible element (allele tm351b), therefore the do not require tamoxifen to allow translocation of the cre recombinase to the nucleus. This strain undergoes cre-mediated recombination of lox sites whenever there is expression driven by the Ple198 MiniPromoter. As per the icre/ER^{T2} mice, any progeny of cells that have undergone recombination will permanently express the tdTomato reporter. We generated this allele by breeding the original tm351a icre/ER^{T2} allele-carrying strain with the C57BL/6-Tg(CAG-Flpo)1Afst/Mmucd strain (MMRRC Stock #032247-UCD), which constitutively and ubiquitously expresses the Flp recombinase and acts on the F3 FRT sites (FIG. 3A).

The Ple198-icre/ER^{T2} (allele tm351a) mice given tamoxifen resulted in tdTomato expression in both the stroma and the epithelium of the cornea (FIG. 3C). In the retina, we observed sparse staining in the ganglion cell layer and the inner nuclear layer, indicative of amacrine and horizontal cells. Although it is unclear which retinal cell types express endogenous Slc6a4, existing evidence suggests a role of serotonin in amacrine, ganglion, and bipolar cells of the retina (Hansson et al. NSC 1999. 89: 243-265; Zhu et al. Vis Neurosci 1995. 12:11-19). The brain was mostly negative apart from some thalamic regions such as the sensory-motor cortex related thalamus and the medial group of the thalamus.

The expression pattern observed with the Ple198-icre mice (allele tm351b) overlapped with the tm351a expression but was also more widespread (FIG. 3D). For example, corneal staining was nearly identical whereas retinal staining contained additional cell types. Staining was observed in putative bipolar cells and Müller glia in the inner nuclear layer, and possibly even some rare photoreceptors in the outer nuclear layer. The brain contained much stronger thalamic expression, in addition to expression throughout the olfactory bulbs, cortex, striatum and hippocampus (close-up shown). Some aspects of the ventral midbrain were also positive.

Table of Sequences

| | | |
|---|---|---|
| SEQ ID | pEMS1136, SLC6A4-D or | SLC6A4_Ple198_full_actual |
| NO: 1 | Ple198 | |
| SEQ ID | pEMS1135, SLC6A4-C or | |
| NO: 2 | Ple197 | |
| SEQ ID | pEMS1133, SLC6A4-A-Prom | SLC6A4_prom_actual |
| NO: 3 | | |
| SEQ ID | SLC6A4-1 or regulatory | |
| NO: 4 | region 1 | |
| SEQ ID | SLC6A4-2 or regulatory | SLC6A4_region2_actual |
| NO: 5 | region 2 | |
| SEQ ID | SLC6A4-3 or regulatory | SLC6A4_region3_actual |
| NO: 6 | region 3 | |

What is claimed is:

1. An isolated polynucleotide comprising a SLC6A4 mini-promoter, wherein the SCL6A4 mini-promoter comprises at least one SLC6A4 regulatory elements with substantial similarity to SEQ ID NO: 4, 5 or 6 operably joined to an SLC6A4 basal promoter with substantial similarity to SEQ ID NO: 3 through a non-native spacing between the regulatory element and the basal promoter.

2. The polynucleotide of claim 1 comprising a SLC6A4 mini-promoter with substantially similarity to SEQ ID NO: 1.

3. The polynucleotide of claim 1 comprising a SLC6A4 mini-promoter with substantially similarity to SEQ ID NO: 2.

4. The isolated polynucleotide of claim 1, operably linked to an expressible sequence.

5. A vector comprising the isolated polynucleotide of claim 1.

6. A cell comprising the vector of claim 5.

7. The cell of claim 6, wherein the vector is stably integrated into the genome of the cell.

8. The cell of claim 6, wherein the cell is a stem cell, a neural progenitor, a neuronal cell, a serotonergic cell, a thalamic brain cell or a retinal cell.

9. A method of expressing a sequence of interest, the method comprising operably linking the sequence of interest to the polynucleotide of claim 1; and introducing into a cell permissive for expression from the SLC6A4 mini-promoter.
